(19) United States
(12) Patent Application Publication
(10) Pub. No.: US 2014/0370588 A1
(43) Pub. Date: Dec. 18, 2014

(54) FILTRATION APPARATUS FOR CONTINUOUS PERFUSION

(71) Applicants: ATMI PACKAGING, INC., Bloomington, MN (US); ARTELIS, S.A., Brusells (BE)

(72) Inventors: Jose Antonio Castillo Gonzalez, Brussels (BE); Vishwas Pethe, Shakopee, MN (US)

(21) Appl. No.: 14/363,825

(22) PCT Filed: Dec. 7, 2012
(86) PCT No.: PCT/US12/68513

§ 371 Date: Jun. 9, 2014

Related U.S. Application Data
(60) Provisional application No. 61/568,872, filed on Dec. 9, 2011.

(51) Int. Cl. C12M 1/00; C12M 1/06

(52) U.S. Cl. CPC C12M 29/04; C12M 27/02; C12M 23/14; C12M 29/06
USPC 435/296.1; 435/297.1; 435/297.2

(57) ABSTRACT

In one aspect, an apparatus for bioprocessing using a liquid comprises an at least partially flexible vessel for receiving the liquid, and a filter adapted for moving within the liquid. An agitator may also be provided for agitating the liquid, and may be connected to the mixer. The mixer may spin or may not spin about an axis of rotation.

This is application claims the benefit of U.S. Provisional Patent Application Ser. No. 61/568,872, the disclosure of which is incorporated herein by reference.

### TECHNICAL FIELD

This disclosure relates generally to the fluid processing arts and, more particularly, to a filtration apparatus for continuous perfusion.

### BACKGROUND OF THE INVENTION

Cells typically require homogenous growth media with optimum levels of oxygen, pH, nutrients (sugar, micronutrients, etc.), and temperature. This may be accomplished in a specially adapted vessel, termed a bioreactor, for housing the cultured cells and media, usually under sterile conditions and usually with a capability for fluid addition, removal, or agitation. Examples of possible vessels for such use may be found in U.S. Patent Application Publication Nos. 2009/0130757, 2010/0190963, and 2010/0015696, as well as U.S. Pat. Nos. 6,544,778, 6,494,613, 7,384,027, and 7,384,873 the disclosures of each of said applications and patents hereby being incorporated by reference in their entireties.

Often, it is desirable to perform extractions from the bioreactor, such as by removing media while leaving the cells intact, and then adding fresh media. This process is generally called “perfusion,” and may occur continuously during the bioprocessing event to ensure that the cells interact with and have a ready supply of fresh media to ensure that proper growth conditions are maintained. While perfusion might be done using a stationary filter to extract the media from the vessel, certain problems are envisioned, including clogging (which is a special concern in many applications given the usually gentle nature of the mixing and the proliferation of cells considered a desirable outcome).

Centrifugal “spin” filters have been used in bioreactors comprised of rigid tanks, which are typically formed of non-corrosive and generally inert stainless steel. These types of filters have not been proposed for use in modem disposable bioreactors comprised of flexible sidewalls, such as bags formed of thin film. This is perhaps because of the lack of reliable support for the relatively heavy and rapidly spinning filter, the inability of the film walls to sustain the resulting torque while maintaining the desirable leak-free condition of the vessel, or the chance of perforation resulting from the high rotational speed of the filter recommended to maximize efficiency (which also generates stresses that negatively impact the process, especially for delicate cell suspensions). Consequently, one approach for achieving filtration in disposable bioreactors is to place a filtration unit outside of the vessel, which is disadvantageous for obvious reasons.

Accordingly, a need is identified for a solution to resolve the foregoing issues, as well as possibly others not yet identified or known to exist. The solution would preferably be easy and inexpensive to implement, and would provide significant advantages over existing filtration apparatus for performing continuous perfusion, and especially in relation to bioreactors. The solution would also be able to be retrofitted onto existing technologies to thereby improve the results achieved, despite the past limitations.

### SUMMARY

One aspect of the disclosure relates to an apparatus for bioprocessing using a liquid. The apparatus may comprise an at least partially flexible vessel for receiving the liquid, and a filter adapted for moving within the liquid in a controlled manner.

In one embodiment, the vessel comprises a plastic bag. In this or other embodiments, the apparatus may further comprise an agitator for agitating the liquid. The filter may be attached directly to an outer surface of the agitator, which may spin or may not spin. In one embodiment, the agitator extends at least partially into a sleeve connected to the vessel. A conduit may also be provided for delivering liquid to or from the filter, wherein the conduit extends at least partially within the sleeve.

In some embodiments, the agitator comprises a paddle. The paddle may be mounted for pivoting movement. In other embodiments, the agitator spins about an axis. A rotatable hub or bearing may be associated with the vessel and the agitator.

The filter may substantially surround the agitator. The filter may also be integral with the sleeve. The filter may comprise a mesh or membrane adapted for allowing the passage of a liquid but not cells.

The filter may be arranged to divide the liquid in an interior compartment of the vessel into a first zone essentially free of cells and a second zone including cells. A first conduit may be provided for removing liquid from the first zone. A second conduit may also be provided for supplying liquid to the second zone.

The apparatus may further include a pump associated with the filter. The pump may be adapted for pumping liquid from the vessel through the filter. Alternatively, the pump may be adapted for pumping liquid through the filter from the vessel.

The apparatus may further include a sparger for introducing a gas into the vessel. The sparger may be connected to the vessel, or to an agitator if present. A filter may also be provided external to an interior compartment of the vessel.

In another aspect, the disclosure pertains to an apparatus for use in processing a liquid. The apparatus comprises a vessel for the liquid having a flexible wall, an agitator for agitating the liquid, and a filter for filtering the liquid. The filter is adapted for attachment to the agitator.

In still another aspect of the disclosure, an apparatus for use in processing a liquid, comprises a vessel for receiving the liquid, an agitator for agitating the liquid, a motive device for connecting with the agitator in the vessel for moving the agitator through the liquid, and a filter for filtering the liquid, said filter configured for attachment to the agitator.

In any of the above-mentioned embodiments or others, the following features may be applied. The vessel may comprise a bag, and the agitator may be associated with a sleeve positioned in a compartment of the vessel. The apparatus may further include a conduit associated with the filter, wherein the conduit is positioned at least partially within the sleeve. The agitator may comprise a paddle, which may be mounted for pivoting movement relative to the vessel.

The filter may comprise a mesh or membrane adapted for allowing the passage of liquid but not cells. The filter may separate a first portion of the liquid including substantially no cells from a second portion of the liquid including cells. A conduit for withdrawing liquid from the first portion and a second conduit for adding liquid to the second portion.

A pump may also be associated with the filter. The pump may be adapted for pumping liquid from the vessel through the filter. Alternatively, the pump may be adapted for pumping liquid through the filter into the vessel. A sparger may also be provided for introducing a gas into the liquid in the vessel.

A further aspect of the disclosure pertains to an apparatus for use in processing a liquid. The apparatus comprises a vessel for receiving the liquid having at least one flexible wall. A movable filter may be provided for filtering the liquid. A motive device may also be provided for moving the filter through the liquid.

The vessel may comprise a flexible bag, and further including a rigid container for supporting the bag. A support structure may also be provided for supporting the motor above the bag.

A further aspect of the disclosure relates to a filtration apparatus for use with a vessel for a liquid. The apparatus comprises a filter for filtering the liquid, and a motive device for moving the filter along a non-linear path throughout the liquid. The apparatus may optionally include a support for supporting the filter above a floor of the vessel. The motive device may include a magnet, and the apparatus may further include an agitator for forming a magnetic coupling with the magnet of the motive device.

Yet another aspect of the disclosure pertains to an apparatus for use in processing a liquid, comprising an at least partially magnetic mixer including at least one blade, said mixer further including a filter for filtering the liquid.

Still a further aspect of the disclosure relates to an apparatus for use in processing a liquid, comprising a mixer having a blade for agitating the liquid, said blade comprising a filter for the liquid.

Another aspect of the disclosure is apparatus for use in processing a liquid. The apparatus comprises a shaft including at least one blade adapted for agitating the liquid, said shaft carrying a filter for filtering the liquid. The shaft may comprise a flexible sleeve, or may be rigid.

A bioreactor may incorporate any or all of these aspects.

Part of this disclosure also relates to a method of handling a liquid. The method comprises providing a filter for filtering the liquid in a vessel adapted for containing the liquid, and providing a shaft in the vessel for connecting with the filter without the shaft contacting the liquid. The step of providing a shaft may comprise inserting the shaft into a sleeve within the vessel. The method may further include the step of moving the shaft and the filter together.

### BRIEF DESCRIPTION OF THE DRAWINGS

FIG. 1 is a schematic view showing the inventive concept in one broad form;

FIG. 2 is a partially schematic, partially perspective view of one possible embodiment made according to the disclosure;

FIG. 3 is a partially schematic, partially perspective view of a mixer for use in accordance with the disclosure;

FIG. 4 is a partially cutaway perspective view of a mixer including a filter;

FIG. 5 is a partially cutaway perspective view of a mixer having a sleeve including an integral filter;

FIG. 6 is a partially cutaway side view of the mixer of FIG. 5;

FIG. 7 is a schematic diagram illustrating one manner of performing continuous perfusion; and

FIGS. 8, 9, and 10 represent partially cutaway side views of other possible embodiments made according to the disclosure.

### DETAILED DESCRIPTION

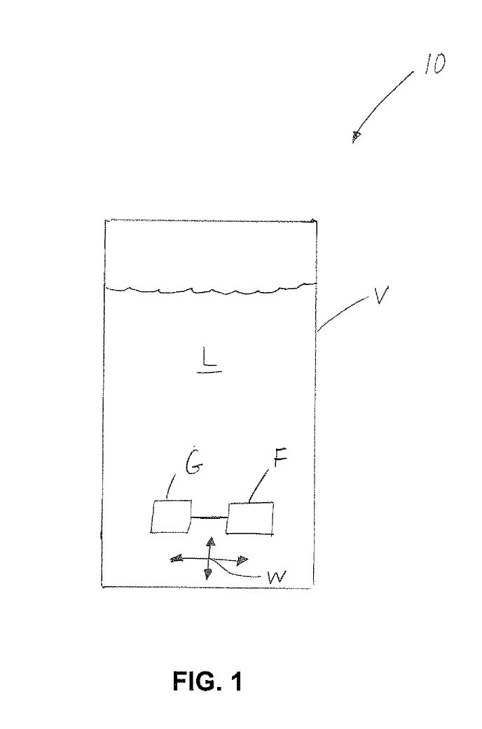

Reference is now made to FIG. 1, which broadly illustrates schematically the inventive concept in one possible form. A system 10 for processing a liquid (which may be considered a growth media) includes a disposable vessel V (which may be a cell culture bioreactor, microbial fermentor or the like) for receiving the liquid L. The vessel V in turn includes an agitator G for agitating the liquid and a filter F for filtering the liquid. The filter F is movable about the container or vessel V in a controlled manner, such as by being connected to the agitator G. Consequently, the movement of one in any direction (e.g., corresponding to vertical and horizontal arrows W) necessarily causes the movement of the other, such that the action of the agitator G in agitating the liquid also results in the filter F providing a filtering function for the liquid.

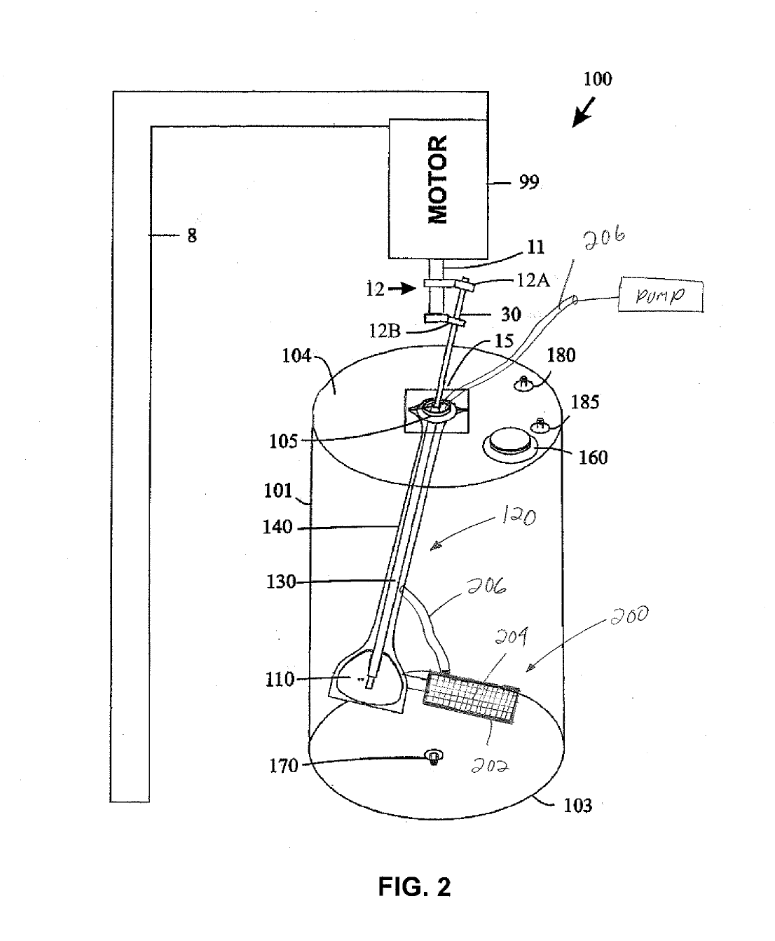

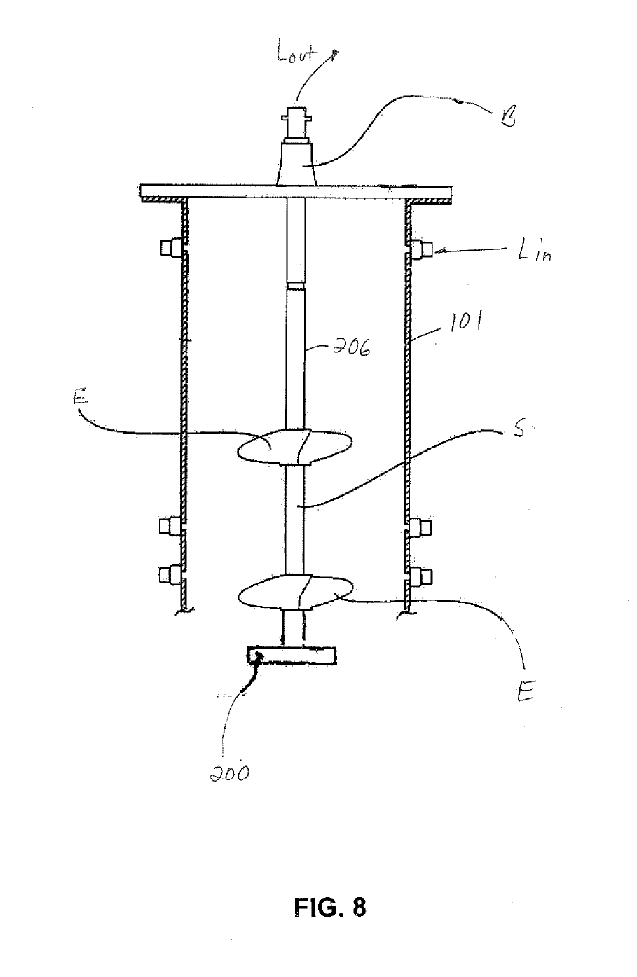

FIG. 2 shows a more detailed implementation of one possible system 100 for processing a liquid, and in particular a liquid containing cells in suspension. The system 100 includes a disposable vessel 101, which could be a rigid plastic tank but is preferably a single-use disposable bag (which most preferably is positioned in an outer rigid support container (see FIG. 8)). However, the vessel 101 may be a bag that is semi-rigid, which may thus include a flexible wall, such as a sidewall, and a rigid wall, such as a bottom wall or floor. While the vessel 101 is illustrated as being generally cylindrical, various shapes (e.g., parallelepiped) may be used, without limitation.

In the illustrated embodiment, the vessel 101 includes a top wall 104 with an inlet 160 and access ports 180, 185. A bottom wall 103 of the vessel 101 includes an outlet or drain 170, which may alternately be used to supply gas (e.g., air or oxygen) to the vessel 101, such as for inflation or oxygenation of the liquid. The vessel 101 may further include a sealed sleeve 140 joined to the vessel 101, such as along the top wall 104.

The system 100 further includes an agitator for agitating a liquid when present in the vessel 101. In one embodiment, the agitator is formed by providing a mixer 120 in the compartment of the vessel 101, such as by inserting a mixing paddle 110 in the sealed sleeve 140. This mixing paddle 110 is joined to a shaft 130, which may be hollow. An intermediate transfer shaft 30 extends through a guide 15 to engage (e.g., by insertion) the hollow shaft 130 at one end. A motor 99 is provided for moving the shaft at the other, and may comprise a rotary output shaft 11.

More specifically describing the particular arrangement shown, a reinforced coupling guide 105 forms an aperture for permitting pivotal movement of the shaft 30. The shaft 30 may link to a radially offset coupling 12 including an upper link 12A and a lower link 12B for engaging the output shaft 11 of a motor 99, which is shown positioned above the vessel 101. The links 12A-12B may include bearings or other rotatable support means to allow the shafts 30, 140 not to rotate about their own axes (in other words, they do not spin) despite being driven through a substantially circular path at a non-zero angle relative to a central vertical axis of the vessel 101. This permits the transfer of kinetic energy from the motor 99 to the paddle 110 without continuous degree rotation of the shafts 30, 140. Such movement without axial rotation of the shafts 30, 130 ensures that the sleeve 140, which is sealed (e.g., welded) to the vessel 101, does not twist or tear, or bind the paddle 110.

The motor 99 may be supported by a support 8, with an extension 9 also supporting the pivot guide 15. If desired, such pivot guide 15 may be eliminated in view of the dual offset links 12A, 12B. Preferably, the support 8 further engages the vessel 101 to ensure general correspondence among the motor 99, transfer shaft 30, and sleeve 140.

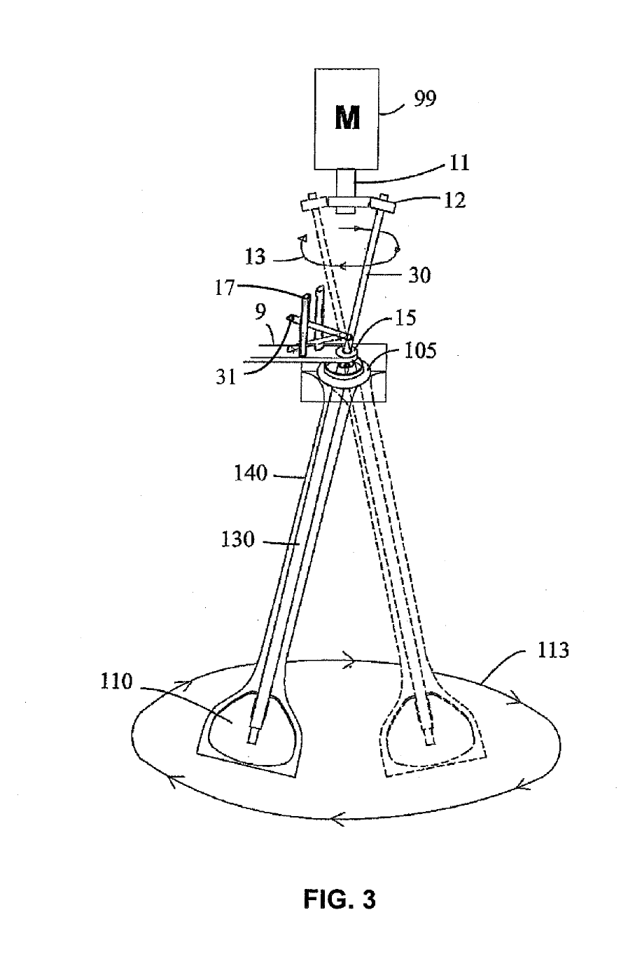

FIG. 3 shows the sealed sleeve 140 and mixing paddle 110 in various positions of a 360° range of motion within the vessel 101. The mixing paddle 110 travels in a large, closed curvilinear (e.g., substantially circular) path 113 in a plane parallel to the bottom surface 103 or base of the vessel 101. Simultaneously, the upper end of the transfer shaft 30 travels in a small, closed curvilinear (e.g., substantially circular) path 13 in a similarly parallel plane, but disposed above the pivot guide 15. The paddle 110 accomplishes such travel without continuous rotation about a longitudinal axis of the shaft 30. Although the offset coupling 12 preferably includes a bearing to enable non-rotation of the rod 30, an anti-rotation rod 31 protruding from the shaft 30 above the pivot guide 15 is retrained between parallel guide bars 17 affirmatively prevents the shaft 30 from rotating about its own longitudinal axis. The travel diameter of the paddle 110 may be modified by adjusting the width of the offset coupling 12, the lengths of the transfer shaft 30 and the hollow shaft 130, and the placement of the pivot guide 15 relative to the shafts 30, 130.

Turning back to FIG. 2, and in accordance with one aspect of the disclosure, a filter 200 may be associated with the system 100. This filter 200 may be provided in the vessel 101 and arranged such that it is capable of moving together with the agitator (e.g., mixer 120), such as by the action of the motor 99 or a like motive device. For instance, as shown in FIG. 2, the filter 200 may include a support 202 that is directly connected to the sleeve 140. The support 202 retains a filter medium 204, such as a fine mesh substrate, that allows for the liquid in the vessel 101 to be drawn through the filter 200. This may be achieved by connecting a conduit 206 to the filter 200, which may include a backing (not shown) defining a compartment for temporarily retaining the liquid once it passes through the medium 204. The conduit 206 may extend to a point external to the vessel 101, such as by passing into and along the sleeve 130, or instead connecting directly to a port, such as ports 180, 185 or even drain 170. In any case, a pump may be associated with the conduit 206 (preferably, external to the vessel 101) for causing the liquid flow through it (and, as discussed below, in either direction).

In any case, movement of the agitator, such as by the pivoting action of the shafts 30, 130 in the illustrated embodiment, moves the filter 200 about the interior of the vessel 101 in a controlled manner. The movement may be along a defined path, with the entirety of the filter 200 bodily moving along this path, without rotating or spinning about its own axis. In other words, the filter 200 may remain stationary relative to the agitator (e.g., mixer including the sleeve 140), but both move together relative to the vessel 101 in a connected or tandem fashion. As should be appreciated, by selectively moving the agitator, such as by controlling the position of the shaft 30, the relative position of the filter 200 within the vessel 101 may be selectively controlled, such as the filtering may be done at a particular zone or location within the vessel at a given point in time.

As can be readily appreciated, this arrangement not only may be used to ensure that the liquid withdrawn through the filter 200 is taken from other than a single, static location within the vessel 101 (as would occur with a static filter), but also helps to prevent clogging of the filter medium 204. In other words, the continuous passage of the liquid over the filter 200 during movement of the agitator helps to prevent the pores or openings in the filter medium 204 from becoming blocked, and thus helps to avoid the need for breaching the sterility to adjust or clean the filter 200. The relatively gentle movement of the filter 200 along the path of travel also avoids the spinning at relatively high speeds required to maximize efficiency with spin filters, and eliminates the concomitant creation of stress, not only in the liquid but also in the working parts of the system 100. Overall, an improved filtration apparatus may be provided for the system 100, and one capable of performing continuous perfusion in an easy and relatively inexpensive manner (thus making it particularly well-adapted to disposable devices).

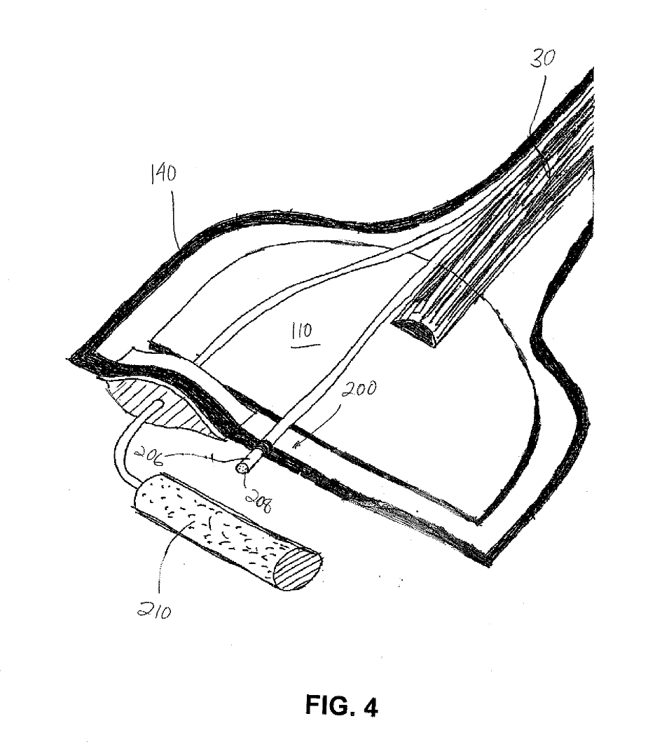

As should be appreciated, the filter 200 may be provided in a different manner while still moving with the agitator, such as the paddle 110 and sleeve combination. For example, as shown in FIG. 4, the filter 200 may be provided directly on or within the end of the conduit 206, such as in the form of a mesh insert 208, or perhaps even a cap (not shown). A device may also be provided for introducing a gas into the liquid, such as a sparger 210, and may be static or, as shown in FIG. 4, coupled to the agitator (e.g., connected to sleeve 140).

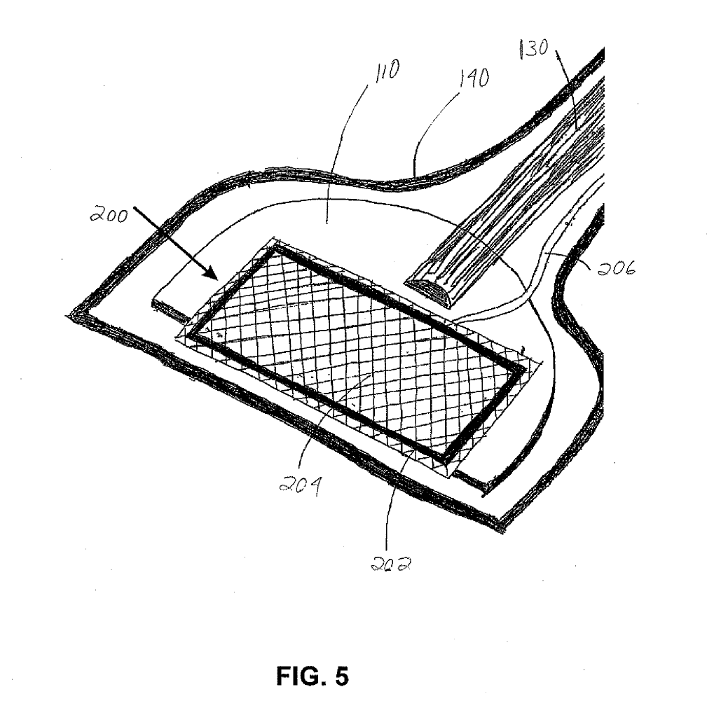

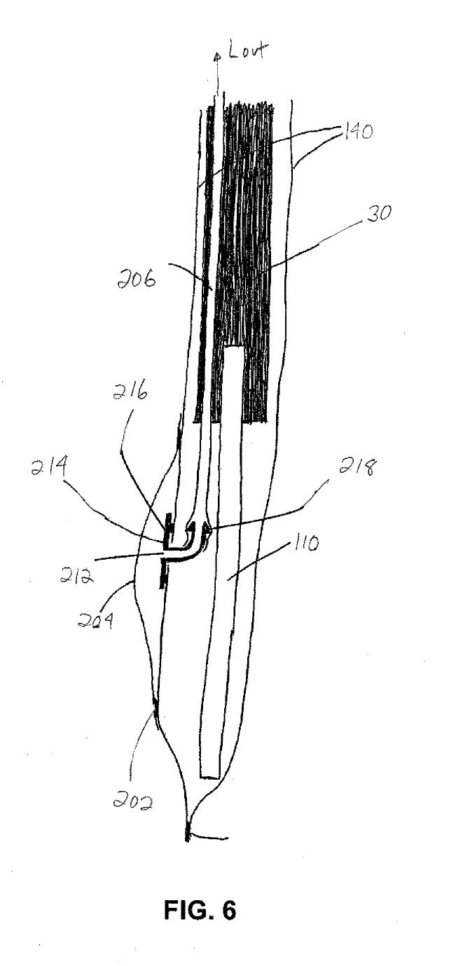

Likewise, as shown in FIGS. 5 and 6, the filter 200 may form an integral part of the sleeve 140. In the illustrated example, the filter medium 204 is connected directly to an external face of the sleeve 140, such as along the portion containing the paddle 110 (but could be provided elsewhere). The attachment may be made by connecting the support 202 directly to the sleeve 140, such as by adhesive or welding. The conduit 206 may received liquid via a port 212 formed by a fitment 214 having a peripheral flange 216 at one end for interfacing with the material of the sleeve 140 and a barb 218 at the other end. The arrangement may be fluid-tight so that sterile conditions, if desired, can be maintained.

As noted above, the pump may be used to withdraw liquid from the vessel 101 via the filter 200. However, it is also possible to reverse the flow, periodically and temporarily, to assist in cleaning the filter medium 204. Of course, the reversed flow may dislodge and eject any debris accumulated on the exposed face of the filter 200, thus helping to ensure optimal filtration.

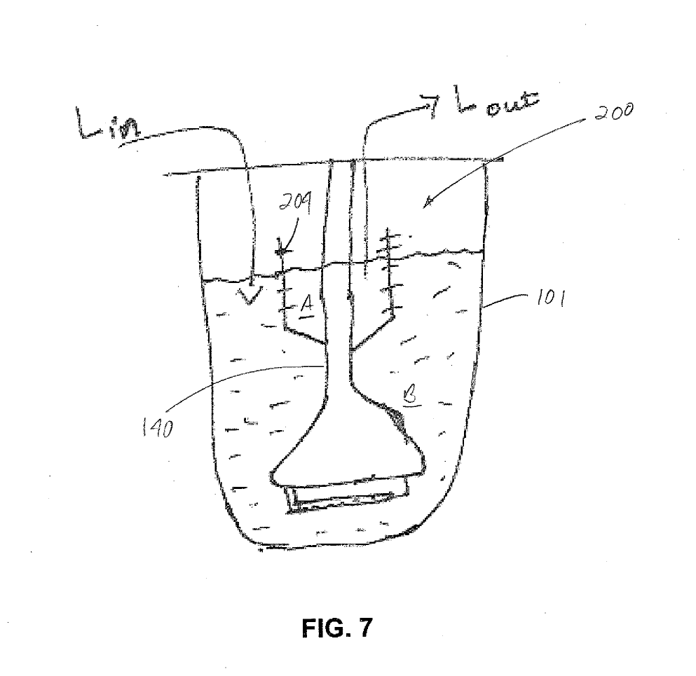

Another embodiment of a system including a filtration apparatus is shown in FIG. 7. The agitator comprises a mixer including the paddle (not shown) and sleeve 140 combination, as outlined in the foregoing discussion. The filter 200 extends along an intermediate portion of the sleeve 140 and essentially forms an open-ended basket, the closed lower boundary of which is defined by a filter medium 204, such as for example a fine (e.g., <100μ) mesh material. The filter medium 204 thus forms two zones: an internal zone A, in which the liquid media can enter free of oversized particles, such as cells, and an external zone B, which includes the liquid media and the cells. As can be appreciated, a perfusion arrangement can be provided such that the medium is extracted from zone A, free from cells or other particles that cannot pass through the filter medium 204, and new liquid media can be introduced to zone B. The circulation created by the agitator thus keeps the liquid travelling between the zones continuously, and thus helps to enhance homogeneity.

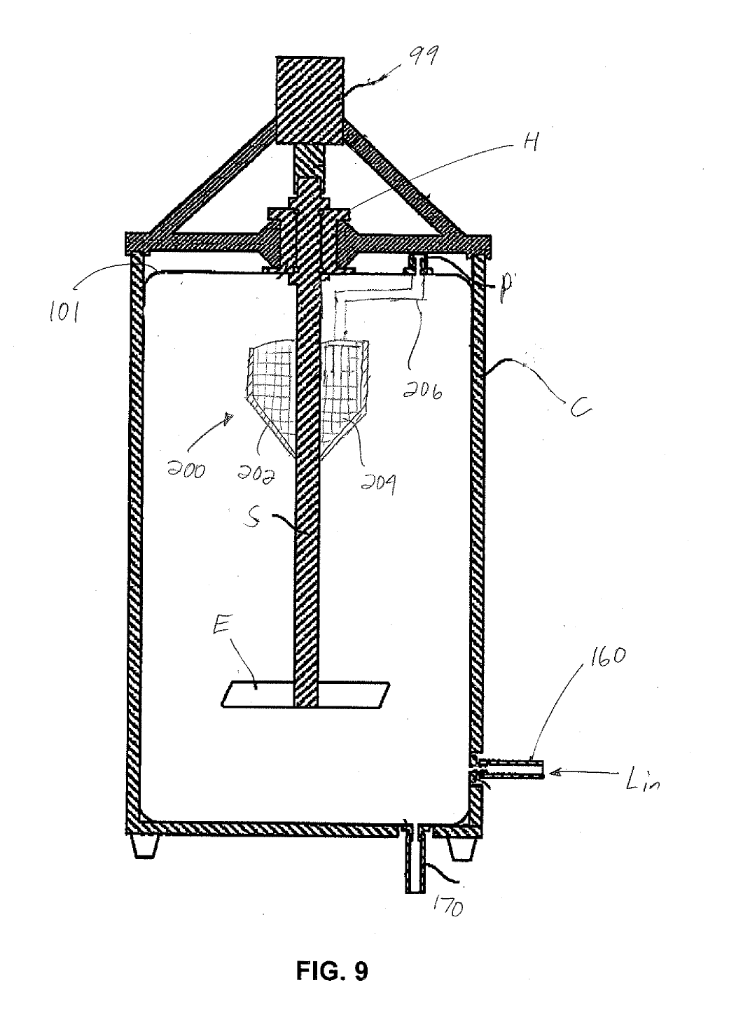

FIGS. 8-9 illustrate alternative embodiments with agitators that do rotate or spin about an axis. In FIG. 8, the filter 200 is connected to a rotatable shaft S carrying one or more blades B. The filter 200 may be carried at the end of the shaft S, which may also serve as the conduit 206 for removing the liquid from the vessel 101 (which as illustrated as a rigid tank with a head plate engaging a bearing B for receiving the shaft S/conduit 206 combination). In other words, the shaft S includes a passage serving as the conduit 206 for transmitting liquid to or from the filter 200.

In FIG. 9, a shaft S carrying a blade B and rotated by an external motor 99 is also illustrated. The shaft S is rotatably supported by a hub H and also carries the filter 200, which includes a filter medium 204 (again, mesh or the like) and associated support 202. The conduit 206 extends between a port P for transmitting liquid and the interior compartment of the filter 200 without being connected to it. Makeup liquid may be supplied through an inlet 160, and a drain 170 may also be provided. The vessel 101 may be disposable, such as a flexible bag supported within a rigid container C.

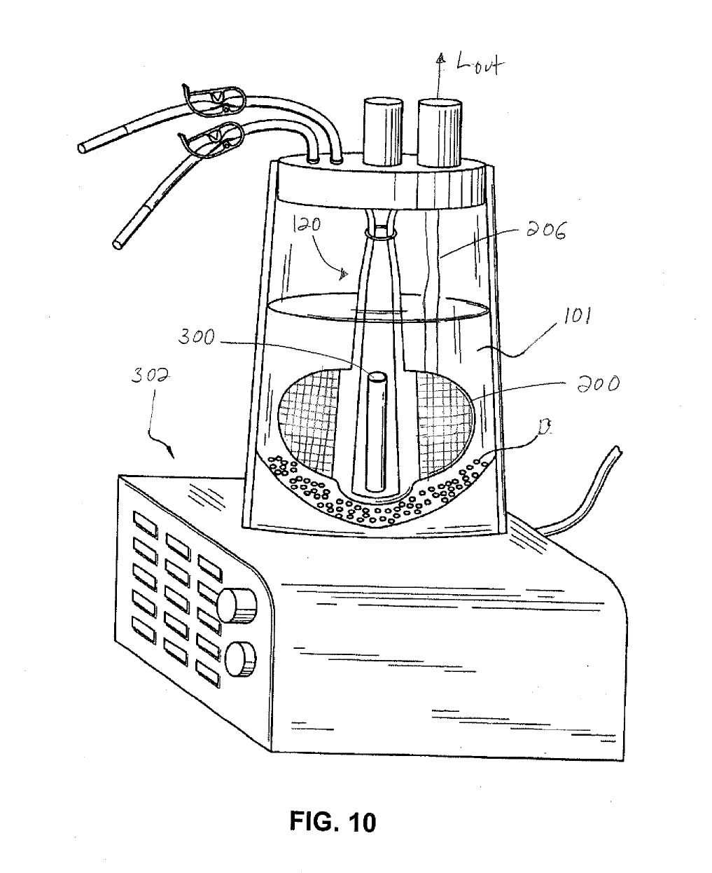

FIG. 10 illustrates a further embodiment with a mixer 120. The mixer 120 may comprise a flexible sleeve 140 that carries the filter 200. Instead of a rotatable shaft 130, a magnet 300 may be provided in the sleeve 140, and an external motive device 302 (such as comprising a motor for moving a magnet) may then be used to form a magnetic coupling that moves the filter 200 about the vessel 101 along a continuous path adjacent the cells (which may be associated with a fluidized or fixed bed D). The conduit 206 extends from the filter 200 to a head plate. As shown, the filter 200 may be positioned to form a blade associated with the mixer, and two filters may be provided with a single mixer, as shown.

The foregoing descriptions of several embodiments made according to the disclosure of certain inventive principles are presented for purposes of illustration and description. The embodiments described are not intended to be exhaustive or to limit the invention to the precise form disclosed and, in fact, any combination of the components of the disclosed embodiments is contemplated. The term “flexible” as used herein in the context of the vessel refers to a structure of the vessel that, in the absence of auxiliary support, may conform to the shape of the liquid contained in the vessel, as contrasted with a “rigid” portion, which retains a pre-determined shape when the liquid is in the vessel. Modifications or variations are possible in light of the above teachings. For example, an agitator that does not spin or pivot, but moves solely in the vertical direction, could be used in place of those shown. In all cases where sterile conditions are desired, it should be appreciated that a suitable connector (perhaps with a filter) may be provided on the conduit 206 external to the interior of the vessel 101. While the filter medium is described as mesh, it should be appreciated that other filter media, such as membranes, thin porous films (e.g., TYVEK), or the like could be used. The filter media may also be disposable, and thus made on an inexpensive material, such as paper or plastic, but it could also be made of metal and possibly even sterilized for re-used. The embodiments described were chosen to provide the best illustration of the principles of the invention and its practical application to thereby enable one of ordinary skill in the art to utilize the invention in various embodiments and with various modifications as are suited to the particular use contemplated. All such modifications and variations are within the scope of the invention when interpreted in accordance with the breadth to which it is fairly, legally, and equitably entitled.

1. An apparatus for bioprocessing using a liquid, comprising:
an at least partially flexible vessel for receiving the liquid; and
a filter adapted for moving within the liquid in a controlled manner.

2. The apparatus of claim 1, wherein the vessel comprises a plastic bag.

3. The apparatus of claim 1, further including an agitator for agitating the liquid.

4. The apparatus of claim 3, wherein the filter is attached directly to an outer surface of the agitator.

5. The apparatus of claim 3, wherein the agitator does not spin.

6. The apparatus of claim 3, wherein the agitator extends at least partially into a sleeve connected to the vessel.

7. The apparatus of claim 6, further including a conduit for delivering liquid to or from the filter, wherein the conduit extends at least partially within the sleeve.

8. The apparatus of claim 3, wherein the agitator comprises a paddle.

9.-12. (canceled)

13. The apparatus of claim 3, wherein the filter substantially surrounds the agitator.

14. The apparatus of claim 6, wherein the filter is integral with the sleeve.

15. The apparatus of claim 1, wherein the filter comprises a mesh or membrane adapted for allowing the passage of a liquid but not cells.

16. (canceled)

17. The apparatus of claim 1, wherein the filter is arranged to divide the liquid in an interior compartment of the vessel into a first zone essentially free of cells and a second zone including cells.

18. The apparatus of claim 17, further including a first conduit for removing liquid from the first zone and a second conduit for supplying liquid to the second zone.

19. The apparatus of claim 1, further including a pump associated with the filter for either pumping liquid from the vessel through the filter or for pumping liquid through the filter to the vessel.

20-21. (canceled)

22. The apparatus of claim 1, further including a sparger for introducing a gas into the liquid.

23. The apparatus of claim 1, further including a filter external to an interior compartment of the vessel.

24.-44. (canceled)

45. An apparatus for use in processing a liquid, comprising an at least partially magnetic mixer including at least one blade, said mixer further including a filter for filtering the liquid.

46. An apparatus for use in processing a liquid, comprising a mixer having a blade for agitating the liquid, said blade comprising a filter for the liquid.

47.-53. (canceled)
